(19) United States
(12) Patent Application Publication
(10) Pub. No.: US 2025/0336475 A1
(43) Pub. Date: Oct. 30, 2025

(54) PREDICTING FUNCTION FROM SEQUENCE USING INFORMATION DECOMPOSITION

(71) Applicant: Board of Trustees of Michigan State University, East Lansing, MI (US)

(72) Inventors: Christoph ADAMI, Okemos, MI (US); Nitash C G, Lansing, MI (US); Arend HINTZE, Falun (SE)

(73) Assignee: Board of Trustees of Michigan State University, East Lansing, MI (US)

(21) Appl. No.: 18/868,262

(22) PCT Filed: Aug. 9, 2023
(86) PCT No.: PCT/US2023/029806

§ 371 Date: Nov. 22, 2024

Related U.S. Application Data
(60) Provisional application No. 63/396,252, filed on Aug. 9, 2022.

(51) Int. Cl. G16B 30/00; G16B 40/20

(52) U.S. Cl. CPC G16B 30/00; G16B 40/20

(57) ABSTRACT

A method of determining the function of a sequence using information decomposition includes providing a plurality of sequences forming a knowledge base, each of the plurality of sequences having respective functions associated therewith, forming a plurality of position weight matrices having different orders based on the sequences, generating a sequence score for each of the plurality of sequences to form a plurality of sequence scores, correlating the respective functions with the sequence scores to form correlation coefficients, selecting a selected order from the different orders based on correlation coefficients, generating a test sequence score for a test sequence based on the selected order and determining a function of the test sequence based on the test sequence score and the knowledge base sequence scores.

### CROSS-REFERENCE TO RELATED APPLICATIONS

This application claims the benefit of U.S. Provisional Application No. 63/396,252, filed on Aug. 9, 2022. The entire disclosure of the above application is incorporated herein by reference.

### FIELD

The present disclosure relates to predicting the function of symbolic sequences and, more particularly to a system and method for predicting the function of a symbolic sequence.

### BACKGROUND

This section provides background information related to the present disclosure which is not necessarily prior art.

In numerous scientific and engineering domains, researchers encounter symbolic sequences whose function is unknown or uncertain. In biology, this could be nucleic acid or protein sequences, while in the neurosciences this could be neural recording trains. In a clinical setting, these sequences could represent genes that may or may not be mutated or otherwise modified, causing disease. Standard approaches attempt to determine the function of these sequences by creating models of the sequence-function relationship. This approach has the drawback that noise in the data is modeled as well, leading to worsening performance when existing data sets are small.

### SUMMARY

This section provides a general summary of the disclosure and is not a comprehensive disclosure of its full scope or all of its features.

The method described here uses information theory to extract the information stored in the sequence, which makes it possible to determine function or functions from sequences without modeling or fitting. Sequences may have multiple functions associated with each sequence (e.g., resistance of a protein to 8 different drugs). The disclosure pertains to a computational method and system that uses information theory to predict the function or functions of symbolic sequences. The disclosure makes it possible to extract information stored in the correlation of multiple variables in a model-free approach, while discarding contributions from noise by leveraging advanced algorithms and statistical techniques. The disclosure takes advantage of the fact that evolution has encoded the function of molecules within sequences, and that the information contained in these sequences makes it possible to predict the function. The disclosure leverages the information decomposition theorem, which proves that information can be decomposed into contributions from monomers, pairs of monomers, triples of monomers, and so on. By extracting information order-by-order, the methods described in this disclosure make it possible to extract only information that has statistical support, while discarding those correlations that are due to chance. The discoveries made through this process have significant applications in various fields, such as biology, genetics, machine learning, and artificial intelligence. Various types of sequences may benefit from the teachings set forth herein. For example, the types of sequences may include but are not limited to nucleic acid sequences, amino acid sequences, neural spike trains, or sequences written in any alphabet. In the following description, multiple sequence alignment is used. However, alignment by motif may be used as well.

The present disclosure has several advantages over existing methods. The method does not involve a modeling or training step, making it computationally simpler than existing methods. The method uses only the information stored in a data set in order to predict a sequence's function, while discarding the noise that is inevitably present in realistic data. This is made possible by decomposing the information stored in sequences into the contribution of single symbols, pairs of symbols, triples of symbols, and so forth. By choosing the order of correlations to include in the determination of function, the researcher can adapt the algorithm to the amount of data they have at their disposal. Further the present system provides for cross-domain applicability. That is, the method's versatility allows its application in various fields, leading to widespread scientific and technological advancements.

In one aspect of the disclosure, a system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. The method also includes providing a plurality of sequences forming a knowledge base, each of the plurality of sequences having a respective function or functions associated therewith, forming a plurality of position weight matrices having different orders based on the sequences, generating a sequence score for each of the plurality of sequences to form a plurality of sequence scores. The method also includes correlating the respective functions with the sequence scores to form correlation coefficients and selecting a selected order from the different orders based on the correlation coefficients. The method also includes generating a test sequence score from a test sequence for the selected order. The method also includes determining a function of the test sequence based on the test sequence score and the sequence scores. Other embodiments of this aspect include corresponding computer systems, apparatus, and computer programs recorded on one or more computer storage devices, each configured to perform the actions of the methods.

Implementations may include one or more of the following features. The method where determining the function of the test sequence may include determining the function of the test sequence using regression. Forming the plurality of position weight matrices having different orders may include determining a first-order position weight matrix and a second-order weight position weight matrix. Forming the plurality of position weight matrices having different orders further may include determining a third-order position weight matrix. Forming the plurality of position weight matrices having different orders further may include determining a greater-than-third-order position weight matrix. Providing the sequences may include one of amino acid sequences, neural spike trains, or sequences written in any alphabet. Providing the knowledge base sequences may include providing nucleic acid sequences. After forming the plurality of position weight matrices, reweighting at least one of the plurality of position weight matrices to remove common ancestry. After forming the plurality of position weight matrices reweighting at least one of the plurality of position weight matrices to resolve ambiguous state assignments. After forming the plurality of position weight matrices reweighting at least one of the plurality of position weight matrices to adjust the strength of selection. Providing the sequences may include one of amino acid sequences, neural spike trains, or sequences written in any alphabet. Implementations of the described techniques may include hardware, a method or process, or computer software on a computer-accessible medium.

One general aspect includes a system with a knowledge base having a plurality of sequences, each of the plurality of sequences having respective functions associated therewith. A controller is programmed to form a plurality of position weight matrices having different orders based on the sequences, generate a sequence score for each of the plurality of sequences to form a plurality of sequence scores, correlate the respective functions with the sequence scores to form correlations, select a selected order from the different orders based on the correlations, generate a test sequence score from a test sequence for the selected order, and based on the test sequence score and the sequence scores, predict the function of the test sequence. Other embodiments of this aspect include corresponding computer systems, apparatus, and computer programs recorded on one or more computer storage devices, each configured to perform the actions of the methods.

Implementations may include one or more of the following features. The system where the controller is programmed to determine the function of the test sequence using regression. The plurality of position weight matrices may include a first-order position weight matrix and a second-order weight position weight matrix. The plurality of position weight matrices may include a first-order position weight matrix, a second-order weight position weight matrix and a third-order position weight matrix. The plurality of position weight matrices may include a first-order position weight matrix, a second-order weight position weight matrix, a third-order position weight matrix and a greater-than-third-order weight matrix. The sequences may include one of amino acid sequences, neural spike trains, or sequences written in any alphabet. The sequences may include nucleic acid sequences. The controller is programmed to reweight at least one of the plurality of position weight matrices to remove common ancestry. The controller is programmed to reweight at least one of the plurality of position weight matrices to resolve ambiguous state assignments. The controller is programmed to reweight at least one of the plurality of position weight matrices to adjust the strength of selection. Implementations of the described techniques may include hardware, a method or process, or computer software on a computer-accessible medium.

One general aspect includes a method including providing a plurality of sequences associated with a case group and a control group. Although two groups are used in this example, more than two groups may be used. The method also includes forming a plurality of position weight matrices having different orders based on the sequences within the case group and the control group. The method also includes generating a plurality of sequence scores for the plurality of position weight matrices to form a plurality of sequence scores. The method also includes generating control histograms and case histograms from the plurality of sequence scores. The method also includes selecting a selected order from the different orders based on the control histograms and the case histograms. The method also includes generating a test sequence score from a test sequence for the selected order. The method also includes classifying the test sequence score as a case sequence or a control sequence based on sequence scores of the selected order. Other embodiments of this aspect include corresponding computer systems, apparatus, and computer programs recorded on one or more computer storage devices, each configured to perform the actions of the methods.

Implementations may include one or more of the following features. The method where classifying may include classifying based on clustering. Forming the plurality of position weight matrices having different orders may include determining a first-order position weight matrix and a second-order weight position weight matrix. Forming the plurality of position weight matrices having different orders further may include determining a third-order position weight matrix. Forming the plurality of position weight matrices having different orders further may include determining a greater-than-third-order position weight matrix. Providing the knowledge base sequences may include providing nucleic acid sequences. After forming the plurality of position weight matrices, reweighting at least one of the plurality of position weight matrices to remove common ancestry. After forming the plurality of position weight matrices reweighting at least one of the plurality of position weight matrices to resolve ambiguous state assignments. After forming the plurality of position weight matrices reweighting at least one of the plurality of position weight matrices to adjust the strength of selection. Implementations of the described techniques may include hardware, a method or process, or computer software on a computer-accessible medium.

One general aspect includes a knowledge base having a plurality of sequences may include case sequences and control sequences and a controller programmed to form a plurality of position weight matrices having different orders based on the sequences within the case group and the control group. The system also includes generating a plurality of sequence scores from the plurality of position weight matrices to form a plurality of sequence scores, generate control histograms and case histograms from the plurality of sequence scores, select a selected order from the different orders based on the control histograms and the case histograms, generate a test sequence score from a test sequence for the selected order, and classify the test sequence score as a case sequence or a control sequence based on sequence scores of the selected order. Other embodiments of this aspect include corresponding computer systems, apparatus, and computer programs recorded on one or more computer storage devices, each configured to perform the actions of the methods.

Implementations may include one or more of the following features. The system where the controller is programmed to classify based on clustering. The controller is programmed to determine the function of the test sequence using regression. The plurality of position weight matrices may include a first-order position weight matrix and a second-order weight position weight matrix. The plurality of position weight matrices may include a first-order position weight matrix, a second-order weight position weight matrix and a third-order position weight matrix. The plurality of position weight matrices may include a first-order position weight matrix, a second-order weight position weight matrix, a third-order position weight matrix and a greater-than-third-order weight matrix. The sequences may include one of amino acid sequences, neural spike trains, or sequences written in any alphabet The sequences may include nucleic acid sequences. The controller is programmed to reweight at least one of the plurality of position weight matrices to remove common ancestry. The controller is programmed to reweight at least one of the plurality of position weight matrices to resolve ambiguous state assignments. The controller is programmed to reweight at least one of the plurality of position weight matrices to adjust the strength of selection. Implementations of the described techniques may include hardware, a method or process, or computer software on a computer-accessible medium.

One general aspect includes a method that includes providing, in a knowledge base, a plurality of sequences having respective sequence scores and a function associated therewith. The method includes generating a test sequence score. The method also includes determining a function of the test sequence based on the test sequence score and the sequence scores. Other embodiments of this aspect include corresponding computer systems, apparatus, and computer programs recorded on one or more computer storage devices, each configured to perform the actions of the methods.

Implementations may include one or more of the following features. The method where determining the function of the test sequence may include determining the function of the test sequence using regression. Forming the plurality of position weight matrices having different orders may include determining a first-order position weight matrix and a second-order weight position weight matrix. Forming the plurality of position weight matrices having different orders further may include determining a third-order position weight matrix. Forming the plurality of position weight matrices having different orders further may include determining a greater-than-third-order position weight matrix. Providing the sequences may include one of amino acid sequences, neural spike trains, or sequences written in any alphabet. Providing the knowledge base sequences may include providing nucleic acid sequences. After forming the plurality of position weight matrices, reweighting at least one of the plurality of position weight matrices to remove common ancestry. After forming the plurality of position weight matrices reweighting at least one of the plurality of position weight matrices to resolve ambiguous state assignments. After forming the plurality of position weight matrices reweighting at least one of the plurality of position weight matrices to adjust the strength of selection. Implementations of the described techniques may include hardware, a method or process, or computer software on a computer-accessible medium.

One general aspect includes a system that also includes a knowledge base having a plurality of sequences, each of the plurality of sequences having respective functions associated therewith, and a controller programmed to generate a test sequence score, and determine a function of the test sequence based on the test sequence score and the sequence scores. Other embodiments of this aspect include corresponding computer systems, apparatus, and computer programs recorded on one or more computer storage devices, each configured to perform the actions of the methods.

Implementations may include one or more of the following features. The system where the controller is programmed to determine the function of the test sequence using regression. The plurality of position weight matrices may include a first-order position weight matrix and a second-order weight position weight matrix. The plurality of position weight matrices may include a first-order position weight matrix, a second-order weight position weight matrix and a third-order position weight matrix. The plurality of position weight matrices may include a first-order position weight matrix, a second-order weight position weight matrix, a third-order position weight matrix and a greater-than-third-order weight matrix. The sequences may include one of amino acid sequences, neural spike trains, or sequences written in any alphabet. The sequences may include nucleic acid sequences. The controller is programmed to reweight at least one of the plurality of position weight matrices to remove common ancestry. The controller is programmed to reweight at least one of the plurality of position weight matrices to resolve ambiguous state assignments. Implementations of the described techniques may include hardware, a method or process, or computer software on a computer-accessible medium.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations and are not intended to limit the scope of the present disclosure.

FIG. 1A is a block diagrammatic view of the case group vs. control group determination system.

FIG. 1B is a high-level block diagrammatic view of the case group vs. control group determination process of FIG. 1A.

FIG. 1C is a flowchart of a method for determining the group determination.

FIG. 2A is a block diagrammatic view of the sequence function determination system.

FIG. 2B is a high-level block diagrammatic view of the function determination process.

FIG. 2C is a flowchart of a method for determining the function of a sequence.

Corresponding reference numerals indicate corresponding parts throughout the several views of the drawings.

### DETAILED DESCRIPTION

Example embodiments will now be described more fully with reference to the accompanying drawings.

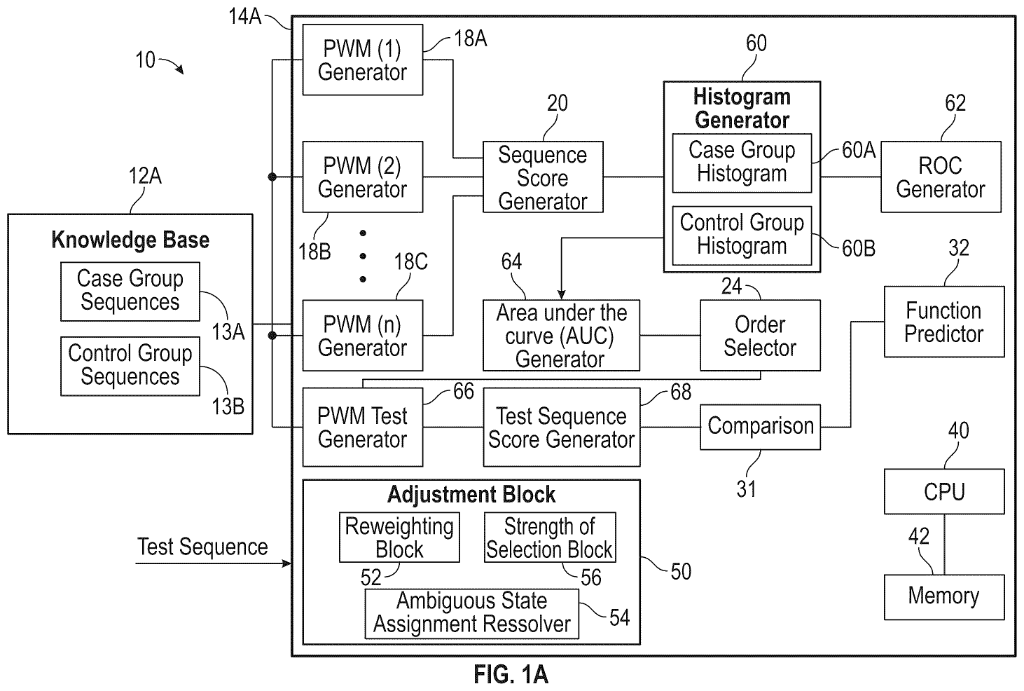

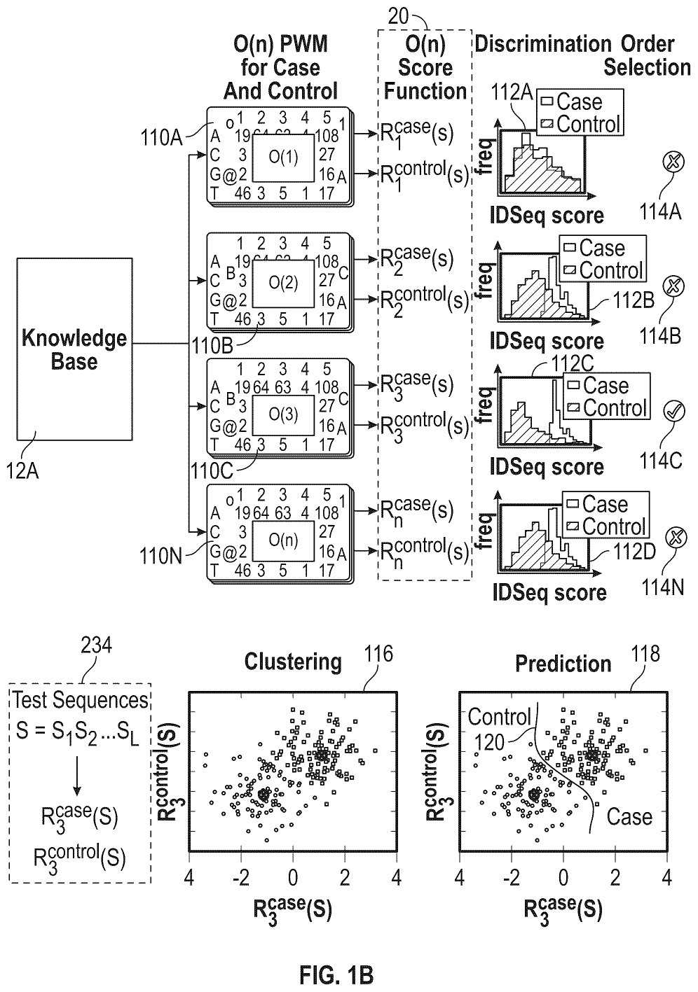

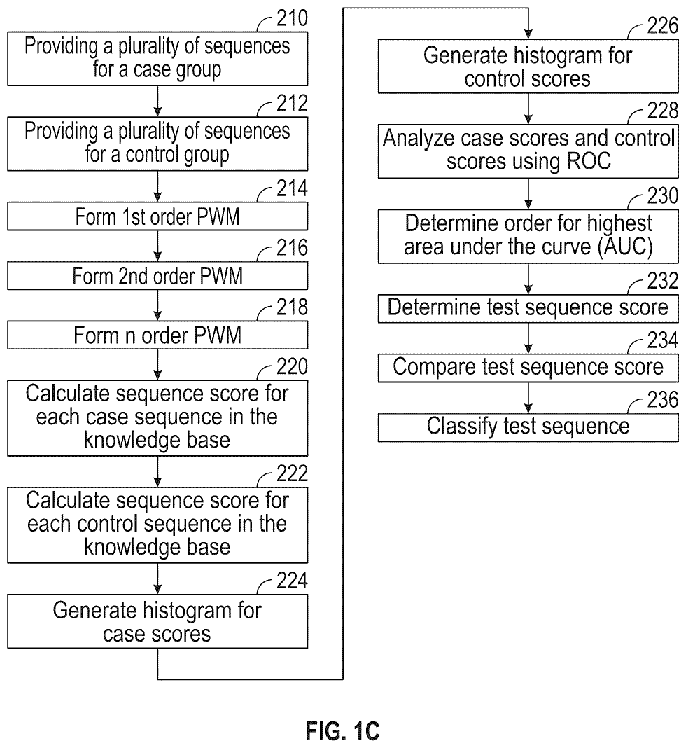

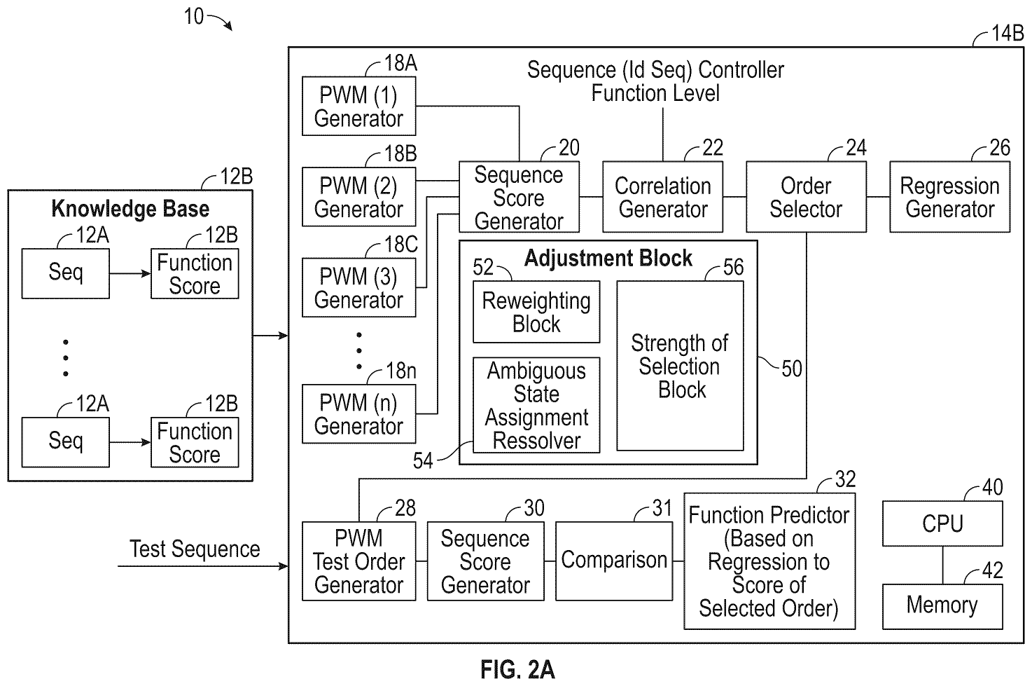

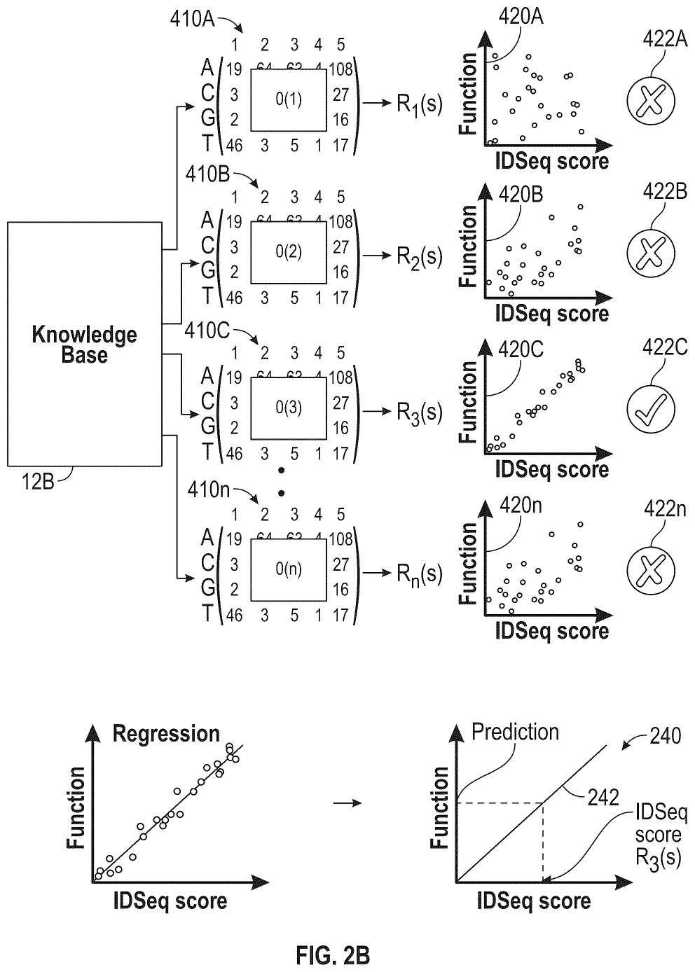

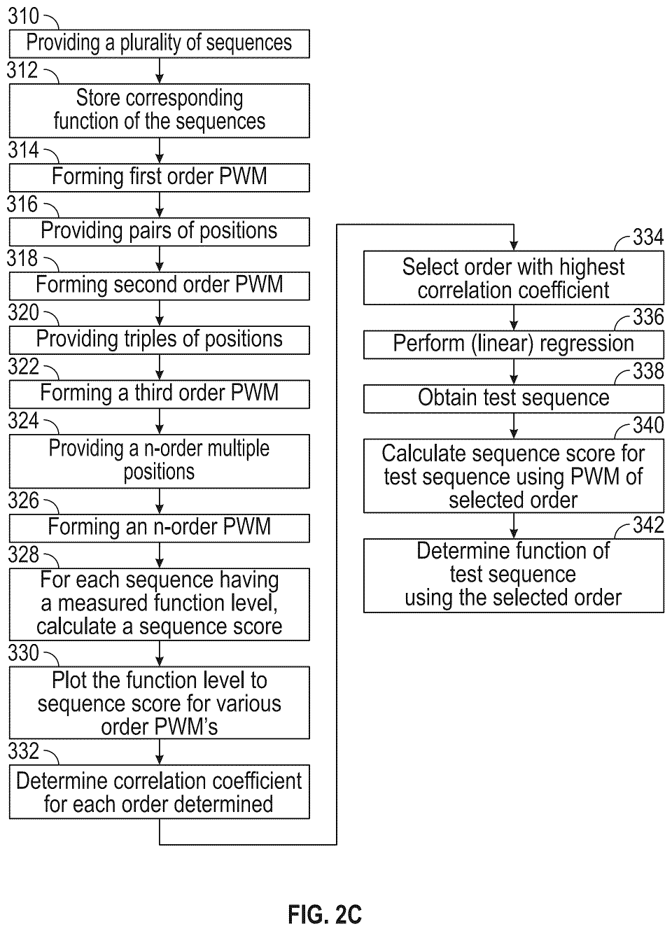

Referring now to FIGS. 1A and 2A, a system 10 and a method set forth herein may be referred to as the “Information Decomposition for Sequences” (IDSeq) system 10 that performs the IDSeq process used to generate a sequence score (IDSeq score or information score) built from position weight matrices (PWMs). The PWMs are built from counting how often a particular pattern of sequence elements appears at a particular position or sets of positions and compare that frequency to an expectation in a comparison block 31. System 10 is ultimately used to determine the function or functions of a test or input sequence in a function predictor 32. There are two types of PWMs: energy matrices and information matrices. Information matrices are described first while energy matrices (giving rise to energy scores) are set forth later. Energy scores may give equal prediction accuracy to information scores.

In FIG. 2B, IDSeq (the system 10) uses a knowledge base 12 (a plurality of example sequences 12A with the target function 12B (or multiple functions)) to predict the function or functions of a sequence that is not contained in the knowledge base (the “test sequence”). In one application of the method, the IDSeq system 12 uses sequences 12A in the knowledge base 12 that have measured functions 12B associated with them. In another application, functions do not have to be provided, as long as it is known that the sequences in the knowledge base are all performing the same function. A sequence controller 14A, 14B calculates information scores for test sequences, and translates these information scores to real-valued functions. In general, high information scores predict superior function, and low information scores predict inferior function. The controller 14A, 14B is programmed to provide a plurality of functions.

In another application of the method shown in FIG. 1A, the IDSeq score or sequences score can be used to classify sequences into different functional classes depending on the information score. Case group sequences 13A and control group sequences 13B are used.

In another application of the method, information scores are replaced by energy scores, where low energy scores predict superior function, and high energy scores predict inferior function.

Information or sequence scores and energy scores are built from position weight matrices (PWMs). The position weight matrices are built from counting how often a particular pattern of symbols appears at a particular set of positions (the position-specific frequency), and compare that frequency to an expectation. The sequence controller 14A, 14B has a plurality of PWM generators 18A, 18B, and 18n. The number of generators 18A-18n may vary. The generators 18A-18n in the figures have a parenthetical that refers to the PWM order, first (1), second (2) up to (n). The order n may in theory extend to the length of the sequence. This may be useful in binary sequences.

First-order energy position weight matrices may be used to predict the efficiency with which transcription factors bind to DNA binding sites, using an energy score function be based on the first-order PWMs. First-order information PWMs for deoxi-nucleic acid (DNA) alphabets have been used. The present disclosure extends this construction to arbitrary alphabets of dimension D. The present disclosure introduces higher-order PWMs, and information score functions of arbitrary order, using the PWMs of arbitrary order. In most cases, first-order estimates of function are not sufficient for real world applications. Including higher-order corrections to the information score increases the precision of prediction to the theoretical maximum: the total amount of information in the knowledge base. According to this, no other method can achieve higher precision.

A typical first-order PWM matrix element is

$${M}_{i , a}^{( 1 )} = {log}_{D} ( \frac{{p}_{i} ( a )}{q i ( a )} ) . \qquad ( 1 )$$

Here, i is the index identifying the position in the sequence, and a numbers the possible states that the symbol can take on at that position. For sequences of length L, the matrix has L columns (the sequence length), and D rows. pᵢ(a) is the maximum likelihood estimator of the probability that the symbol indexed by a appears at position i of the sequence, given by

$${p}_{i} ( a ) = \frac{{n}_{i} ( a )}{N} , \qquad ( 2 )$$

where nᵢ(a) is the number of times symbol a was observed at position i among the N sequences in the knowledge base, and Nis the number of sequences in the knowledge base. In case there are vanishing counts nᵢ(a), the method uses the corrected pᵢ(a) using a first-order pseudocount π₁:

$${p}_{i} ( a ) = \frac{{n}_{i} ( a ) + {π}_{1}}{N + D {π}_{1}} , \qquad ( 3 )$$

The pseudocount is a variable that is chosen by the investigator to match the size of the knowledge base. A starting choice could be π=1/N, but typically a suitable π is chosen by the investigator via optimization.

qᵢ(a) is the a priori expectation for the probability that the symbol indexed by a appears in the sequence at position i for a sequence that is non-functional. There are several different ways to estimate qᵢ(a), depending on the application. In some applications, qᵢ(a) is the uniform distribution over sequence symbols, in which case qᵢ(a)=1/D. In other applications, qᵢ(a) is the likelihood p̅ᵢ(a) that symbol a appears anywhere in any sequence of this type (an “alphabet bias. In this application, alphabet bias can be introduced for arbitrary alphabets, and arbitrary-order PWMs.

In another application, qᵢ(a) refers to the probability that the symbol indexed by a appears at position i of the sequence for a set of sequences that perform a baseline function, and pᵢ(a) refers to the probability that the symbol indexed by a appears at position i of the sequence for a set of sequences that performs an extended function. In that case, the position weight matrix formed using Eqn. (1) quantifies the function of a sequence over-and-above the baseline function. In that manner, the sequence (IDSeq) controller 14A, 14B can quantify relative information. The average of Eqn. (1) (averaged over the sequences in the knowledge base) is equal to the Kullback-Leibler distance of the probability distributions pᵢ(a) and qᵢ(a)

$${M}_{i , a}^{( 1 )} 〉 = {D}_{K L} ( p \vert \vert q ) = {\sum }_{i} {\sum }_{a} {p}_{i} ( a ) {log}_{D} ( \frac{{p}_{i} ( a )}{{q}_{i} ( a )} ) . \qquad ( 4 )$$

A typical second-order PWM is defined as

$${M}_{ij , ab}^{( 2 )} = {log}_{D} ( \frac{{p}_{j} ( a , b )}{{q}_{i} j ( a , b )} ) . \qquad ( 5 )$$

Here, pᵢⱼ(a, b) is the maximum likelihood estimator of the probability that the symbol combination a, b appears at positions i, j of the sequence

$${p}_{i j} ( a , b ) = \frac{{n}_{i} j ( a , b ) + {π}_{2}}{N + {D}^{2} {π}_{2}} , \qquad ( 6 )$$

where π₂ is a second-order pseudocount. Typically, second-order pseudocounts are a factor 1/D smaller than the first-order pseudocount, i.e., π₂≈π₁/D.

In Eq. (5), qᵢⱼ(a, b) is the probability to find symbol combination a, b at positions i, j for a non-functional sequence. In one application, qᵢⱼ(a, b) is given by the uniform distribution of symbols, so that qᵢⱼ(a, b)=1/D². In another application, qᵢⱼ(a, b) is given by the alphabet bias p̅(a)p̅(b). In another application, qᵢⱼ(a, b) refers to the maximum likelihood estimator of the probability that the symbol combination a, b appears at positions i, j of a set of sequences with baseline function that is compared to the target function.

The second-order PWM has

$$( \begin{matrix}L \\ 2\end{matrix} ) = L ( L - 1 ) / 2$$

columns and D² rows. D² is the number of possible pair-motifs, and L(L−1)/2 is the number of all pairs of positions for which i<j. The third-order PWM is defined as

$${M}_{i jk , abc}^{( 3 )} = {log}_{D} \frac{{p}_{ijk} ( a , b , c )}{{q}_{ijk} ( a , b , c )} . \qquad ( 7 )$$

This matrix has

$$( \begin{matrix}L \\ 3\end{matrix} ) = L ( L - 1 ) ( L - 2 ) / 6$$

columns and D³ rows. The probabilities pᵢⱼₖ(abc) are the maximum likelihood estimators to find the combination of symbols a, b, c at positions i, j, k

$${p}_{ijk} ( abc ) \approx \frac{{n}_{ijk} ( a , b , c ) + {π}_{3}}{N + {D}^{3} {π}_{3}} , \qquad ( 8 )$$

where nᵢⱼₖ(a, b, c) counts how many times across the N sequences in the knowledge base the symbol combination abc was observed. The third-order pseudocount is typically π₃≈π₂/D.

An n-th order PWM has

$$( \begin{matrix}L \\ n\end{matrix} )$$

columns and Dⁿ rows, and is constructed in the same manner as Eqs. (1, 5, 7).

The first-order energy PWM is defined as

$${E}_{i , a}^{( 1 )} = {log}_{D} ( \frac{{p}_{i} ( 0 )}{{p}_{i} ( 0 )} ) . \qquad ( 9 )$$

Here, pᵢ(a) refers to the likelihood that symbol a appears at position i of the sequence as defined above, and pᵢ(0) refers to the likelihood that the most common symbol among the D symbols appears at position i (the consensus symbol). For this reason, pᵢ(0)≥pᵢ(a) for any a, and therefore E_{i,a}⁽¹⁾≥0. Higher-order energy PWMs can be constructed according to the way higher-order information PWMs are constructed. For example, the third-order energy PWM is defined as

$${E}_{ijk , abc}^{( 3 )} = {log}_{D} \frac{{p}_{ijk} ( 0 )}{{p}_{ijk} ( a , b , c )} , \qquad ( 10 )$$

where pᵢⱼₖ(0) refers to the likelihood that the most common combination of symbols a, b, c appears at positions i, j, k of the sequences in the knowledge base (the consensus likelihood). The consensus likelihood pᵢⱼₖ(0) is different from the product of the consensus probabilities pᵢ(0)pⱼ(0)pₖ(0) in most cases.

Information PWMs are used to construct information score functions. Energy PWMs are used to construct energy score functions. The information score to order i for a test sequence s=s₁ . . . s_{L} is obtained by noting the combination of symbols that appear at a combination of positions in the sequence, and finding the corresponding entry in the PWM of that order, and adding that entry to the overall score. Mathematically, this can be done by creating sequence matrices of a specific order for the test sequence s. Specifically, the sequence matrix of order 1, S⁽¹⁾, is defined for sequence s=s₁ . . . s_{L} as

$${{S}^{( 1 )} ( \vec{S} )}_{ia} = {δ}_{ia} , \\ where \qquad ( 11 )$$

$${δ}_{ia} = \{ \begin{matrix}1 if {s}_{i} = a , \\ 0 if {s}_{i} \neq a .\end{matrix} \qquad ( 12 )$$

For example, the 9-mer DNA sequence \vec{s}=AATCGGATA corresponds to the first-order sequence matrix

$$\begin{matrix}1 & 2 & 3 & 4 & 5 & 6 & 7 & 8 & 9\end{matrix} \\ {S}^{( 1 )} ( \vec{s} ) = \begin{matrix}A \\ C \\ G \\ T\end{matrix} ( \begin{matrix}1 & 1 & 0 & 0 & 0 & 0 & 1 & 0 & 1 \\ 0 & 0 & 0 & 1 & 0 & 0 & 0 & 0 & 0 \\ 0 & 0 & 0 & 0 & 1 & 1 & 0 & 0 & 0 \\ 0 & 0 & 1 & 0 & 0 & 0 & 0 & 1 & 0\end{matrix} ) \qquad ( 13 )$$

The sequence scores for all of the orders may be generated in a sequence score generator 20. Of course, individual sequence score generators may be used for each order PWM. The first order score R₁(s) is the trace over the product of the PWM and the sequence matrix (here T stands for transposition)

$${R}_{1} ( \vec{s} ) = Tr ( {M}^{( 1 )} {S}^{( 1 ) T} ) = {\sum }_{i = 1}^{L} {\sum }_{a = 1}^{D} {M}_{i , a}^{( 1 )} {S}_{a , i}^{( 1 )} \qquad ( 14 )$$

To obtain the second-order score, the sequence S is translated into the second-order sequence matrix, by defining

$${S}^{( 2 )} \vec{s} = {δ}_{ij , ab} , \\ Where \qquad ( 15 )$$

$${δ}_{ij , ab} = \{ \begin{matrix}1 if {s}_{i ,} {s}_{j} = ab , \\ 0 if {s}_{i} {s}_{j} \neq ab .\end{matrix} \qquad ( 16 )$$

For the example sequence AATCGGATA, the first five columns of the second-order sequence matrix are

$$\begin{matrix}1 , 2 & 1 , 3 & 1 , 4 & 1 , 5 & ⋯\end{matrix} \\ {S}_{2} ( \vec{s} ) = \begin{matrix}AA \\ A C \\ AG \\ AT \\ GA \\ GC \\ GT \\ ⋮ \\ TT\end{matrix} ( \begin{matrix}1 & 0 & 0 & 0 & ⋯ \\ 0 & 0 & 1 & 0 & ⋯ \\ 0 & 0 & 0 & 1 & ⋯ \\ 0 & 1 & 0 & 0 & ⋯ \\ 0 & 0 & 0 & 0 & ⋯ \\ 0 & 0 & 0 & 0 & ⋯ \\ 0 & 0 & 0 & 0 & ⋯ \\ ⋮ & ⋮ & ⋮ & ⋮ & ⋮ \\ 0 & 0 & 0 & 0 & \end{matrix} ) \qquad ( 17 )$$

The second-order score function is

$${R}_{2} ( \vec{s} ) = Tr ( {M}^{( 2 )} {S}^{( 2 ) T} ) = {\sum }_{i < j} {\sum }_{a , b} {M}_{ij , ab}^{( 2 )} {S}_{a , b , ij}^{( 2 )} \qquad ( 18 )$$

The third-order score function is

$${R}_{3} ( \vec{s} ) = Tr ( {M}^{( 3 )} {s}^{( 3 ) T} ) , \qquad ( 19 )$$

and so on for higher-order score functions.

The score functions generated by the sequence score generator 20 are good proxies for the functional information within the sequence, but they are non-trivial. Generally speaking, the Shannon information content of single sequences is undefined because this is only defined groups of sequences like those in the knowledge base 12. If pᵢ(a) is the likelihood to find symbol a at position i for the sequences in the knowledge base, then we can define the first-order entropy of position i as

$$H ( i ) = - {\sum }_{a} {p}_{i} ( a ) {log}_{D} {p}_{i} ( a ) . \qquad ( 20 )$$

The first-order entropy of the knowledge base 12 is the sum over all the positions i

$${H}_{1} = {\sum }_{i = 1}^{L} H ( i ) \qquad ( 21 )$$

The first-order information is then just

$${I}_{1} = L - {H}_{1} \qquad ( 22 )$$

as it is the maximal entropy (given by the sequence length if the logarithm is taken to the base of the alphabet D) minus the actual entropy.

The information score R₁(s) is constructed in such a manner that the Shannon information approximated to first-order I₁ is the average of the first-order PWM (1), that is, the average first-order IDSeq score, averaged over the sequences in the knowledge base 12 (when the unselected distribution qᵢ(a) is given by the uniform distribution 1/D).

$${I}_{1} = 〈 {M}^{1} 〉 = {\sum }_{i , a} {p}_{i} ( a ) {M}_{i , a}^{( 1 )} \qquad ( 23 )$$

But the second-order Shannon information I₂ is not the average of the second-order PWM (the average second-order IDSeq score of sequences in the knowledge base) because (for the case qᵢⱼ(ab)=1/D²)

$$〈 {M}^{( 2 )} 〉 = {\sum }_{i j , a , b} {p}_{i j} ( a , b ) {M}_{i , a}^{( 2 )} = {\sum }_{ij , a , b} {p}_{\dot{ι} j} ( a , b ) ) {log}_{D} ( {D}^{2} {p}_{i j} ( a , b ) ) = L ( L - 1 ) + {\sum }_{ij , a , b} {p}_{i j} ( a , b ) ) {log}_{D} {p}_{\dot{ι} j} ( a , b ) ) . \qquad ( 24 )$$

The last term in Eqn. (24) is minus the joint entropy of all pairs

$${\sum }_{ij , a , b} {p}_{i j} ( a , b ) {log}_{D} {p}_{i j} ( a , b ) ) = - {\sum }_{i < j} H ( ij ) \qquad 25 )$$

so that

$$〈 {M}^{( 2 )} 〉 = L ( L - 1 ) - {\sum }_{i < j} H ( i j ) \qquad ( 26 )$$

However, this is not equal to the second-order approximation to the Shannon information of sequences in the knowledge base. This can be seen by calculating the second-order Shannon information content I₂ using Fano's entropy decomposition theorem

$$H ( {s}_{1} … {s}_{L} ) = {\sum }_{i = 1}^{L} H ( i ) - {\sum }_{i < j}^{L} I ( i : j ) + {\sum }_{i < j < k}^{L} I ( i : j : k ) - … {( - 1 )}^{L - 1} I ( 1 : … : L ) \qquad ( 27 )$$

Here, H(i) is the per-site entropy defined in Eq. (20), I(i:j) is the shared Shannon entropy between sites i and j (a.k.a, the information that site i has about site j), and I(i:j:k) is the information shared between sites i, j, and k, etc. All of the information can be written in terms of joint entropies. This implies that the second-order approximation to the Shannon entropy of sequences in a knowledge base is obtained by neglecting the third-order and higher terms in Eqn. (27) is

$${H}_{2} = {\sum }_{i} {H}_{i} - {\sum }_{i < j} I ( i : j ) = {\sum }_{i} {H}_{i} - {\sum }_{i < j} ( H ( i ) + H ( j ) - H ( i j ) ) = {\sum }_{i} {H}_{i} - ( L - 1 ) {\sum }_{i} H ( i ) + {\sum }_{i < j} H ( ij ) = ( 2 - L ) {H}_{1} + {\sum }_{i < j} H ( i j ) \qquad ( 28 )$$

where H₁ is given by Eqn. (21). The Shannon information to order up to 2 of sequences in the knowledge base therefore is

$${I}_{2} = L - {H}_{2} = L - {\sum }_{i < j} H ( ij ) + ( L - 2 ) {H}_{1} . \qquad ( 29 )$$

When compared to the average of the second-order PWM (averaged over sequences in the knowledge base) shown in Eq. (26) then

$$〈 {M}^{( 2 )} 〉 = {I}_{2} + ( L - 2 ) {I}_{1} \qquad ( 30 )$$

where I₁=L−H₁ is the Shannon information content approximated to first order. Therefore, the average second-order IDSeq score, which is the average of the second-order PWM, is equal to the second-order Shannon information I₂ plus a term proportional to the first order Shannon information. Similar formulae hold for higher-order information. Creating information score functions in such a way that the average information score to a particular order equals the Shannon information to that order does not work because of a conflict between pseudocounts of different orders. The information scores defined in this application only use PWMs of the same order, although they contain information about smaller orders implied by Eq. (30).

In FIGS. 1A, 1B and 1C, the controller 14A further details are set forth below for a method for predicting functional class (case or control) of a test sequence.

The PWM generators 18A-18n are used to generate case and control PWMs. In the first example set forth below, the PWMs may be generated for a case group (case PWMs) and a control group (control PWMs) which are ultimately used for testing a test sequence or group of test sequences. The case group and control group have a common characteristic such as with a disease in the case group or without the disease in the control group. In contrast, FIGS. 2A-2C shown the PWMs generated for sequences each having a known function or functions in the knowledge base 12B and controller 14B. Multiple functions may be associated with each sequence.

The sequence score generator 20 and the details of the sequence score generator 20 are described above.

Ultimately, the controller 14A has a CPU 40 running multiple threads that is used for the performing the process. Although only one CPU 40 is illustrated, multiple CPUs may be used to perform the computation set forth herein. Although only one CPU 40 is illustrated, multiple CPUs or Graphics Processing Units (GPUs) running multiple threads could be used. Likewise, a memory 42 is also illustrated. Other types of memories and distributed memories may be used to store intermediate processes as well as the ultimate functions provided. As mentioned briefly above, the knowledge base 12 may also be incorporated within the memory 42.

An adjustment block 50 may also be provided. The adjustment block 50 may have various components that are used to make adjustments to provide increased accuracy for the function prediction. The functions of each of the blocks of the adjustment block 50 is described in further detail below. In general, the adjustment block 50 may include a reweighting block 52, an ambiguous state assignment resolver 54 and a strength of selection block 56, all of which are described in greater detail below.

In FIGS. 1A, 1B and 1C, one application of the method of the IDSeq system 10 may be used to classify a sequence as belonging to a case group 13A (e.g., presence of disease) or a control group 13B (e.g., healthy patients). The sequence to be classified may be referred to as test sequence. In step 210, the knowledge base 12 having case group sequences 13A are provided. In step 212 control group sequences 13B are provided. Only two sequence groups are shown in FIGS. 1A-C and 2A-C, but the method allows for an arbitrary number of groups. Each group has a functionality in common.

PWMs of ascending order are created for both the case group sequences 13A and the control group sequences 13B as illustrated in 110A-110n. In this example, step 214 generates first-order PWMs, step 216 generates second-order PWMs and step 218 generates nth-order PWMs, up to the desired order. In step 220 the sequence score generator 20 generates the sequence score (the IDSeq score) for each order and for each case sequence 13A and for each control sequence 13B in the control sequences 13B in the knowledge base 12. In step 224, a histogram for case scores is generated by the histogram generator 60 as a case group generator 60A in a control group histogram generator 60B. In step 226 a histogram for control scores is generated. The histograms 112A, 112B 112C and 112n are shown in FIG. 1B.

In step 228 the case and control scores are analyzed via receiver operating characteristic (ROC) curves using the histograms in the ROC generator 62. An ROC curve (receiver operating characteristic curve) is an indicator graph showing the performance of the classification model. In step 230, the sequence score (IDSeq order) based on the PWMs with the highest area under the curve (AUC) of the ROC determined in the AUC generator 64 is selected to perform discrimination, but other machine learning methods can be used as well. In this example, the histogram 112C is selected with the highest area under the curve in the order selector 24. In step 232, the order with the highest correlation coefficient is selected in the order selector 24 and the PWM test generator 66 uses the PWM order selected for evaluating the test sequence. As indicated by the selection block 114C being a check mark (indicating the selected one) versus the selection blocks 114A, 114B and 114n being Xs.

In step 234, a test sequence score is generated at the test sequence score generator 68 based on the selected PWM from the knowledge base based on the selected order (as indicated PWM test generator 66). Test sequence scores are evaluated or compared relative to both the case and the control in step 234 at the comparison block 31, and the position in a two-dimensional clustering plot 116 of case score vs. control score is analyzed. A machine learning clustering algorithm (for example k-nearest neighbor clustering, but any appropriate clustering algorithm can be used) determines whether the test sequence should be classified as a case sequence or control sequence in the step 236 and in the plot 118 by the function predictor 32, which indicates the case or the function or functions of the test sequence. Datasets may have multiple functions associated with each sequence (e.g., resistance of a protein to 8 different drugs). A plotted line 120 may be used to divide the control sequences from the case sequences. By determining the region of the plot 118, the test sequence is classified.

Referring now to FIGS. 2A, 2B and 2C a method for predicting functional activity level of a test sequence is set forth.

The PWM generators 18A-18n and the sequence score generated in the sequence score generator 20 are described above.

In general, a correlation generator 22 determines a correlation as described in more detail below. Based on the highest correlation between scores and functions for sequences in the knowledge base, an order selector selects the order to be used to evaluate the test sequence score. The PWM test generator 28 selects the PWM from the PWM generators that has selected order to be used in the comparison with the test sequence score from the test sequence score generator 30. The order selector communicates with a regression generator 26. A test sequence is generated or communicated to the controller 14. Ultimately, a test sequence score generator 30 generates a test sequence score and, in coordination with a function predictor 32, generates the prediction of the function based on the selected order. Details of this is set further below.

In order to predict the level of functional activity of a test sequence, the method uses a knowledge base in which the functional activity level of example sequences has previously been determined experimentally. The system 10 and method uses this knowledge base to predict the functional activity of a test sequence using regression of the information score. In preparation for this step, a weight has to be assigned to each sequence in the knowledge base 12B, where the weight is proportional to the sequence's activity level. The purpose of this step is to simulate a population in which sequences of higher activity have a higher prevalence, because the theoretical basis of PWMs requires that the sequences are in mutation-selection balance. If sequences in a knowledge base 12B are assigned a weight wⱼ, then the sum of the weights of the sequences in the knowledge base defines the effective knowledge base size

$${\sum }_{j = 1}^{N} {w}_{j} = {N}_{eff} \qquad ( 31 )$$

When counting the number of times a symbol a appears at position i in the knowledge base 12B, the count now adds the weight of the sequence

$${\tilde{n}}_{i} ( a ) = {\sum }_{j = 1}^{N} {w}_{j} {δ}_{a , x ( i , j )} . \qquad ( 32 )$$

where ñᵢ(a) is the weighted count. In Eq. (32), x(i, j) is the symbol found at position i for the jth sequence in the knowledge base and δ_{a,x(i,j)}=1 only when residue a appears at position i in the jth sequence in the knowledge base.

This weighted count is used to calculate the weighted maximum-likelihood estimate

$${\tilde{p}}_{i} ( a ) = \frac{{\tilde{n}}_{i} ( a )}{{N}_{eff}} , \qquad ( 33 )$$

which in turn is used to construct the weighted first-order PWM

$${\tilde{M}}_{i , a}^{( 1 )} = {log}_{D} \frac{{\tilde{p}}_{i} ( a )}{{q}_{i} ( a )} . \qquad ( 34 )$$

Higher-order weighted PWMs are constructed in the same manner. For example, the second-order weighted PWM is given by

$${\tilde{M}}_{i , j , ab}^{( 2 )} = {log}_{D} \frac{{\tilde{p}}_{ij} ( a , b )}{{q}_{i j} ( a , b )} \qquad ( 35 )$$

with

$${\tilde{p}}_{i j} ( a , b ) = \frac{{\tilde{n}}_{ij} ( a b )}{{N}_{eff}^{( 2 )}} , \qquad ( 36 )$$

$$where {N}_{eff}^{( 2 )} = {\sum }_{a b} {\tilde{n}}_{i j} ( ab ) .$$

Using weights to approximate a sequence's prevalence in the knowledge base 12B, the weight of sequence j in the knowledge base 12B with annotated activity level fⱼ should be adjusted to

$${w}_{j} = \frac{{f}_{j}^{v}}{{\sum }_{k = 1}^{N} {f}_{k}^{v}} \qquad ( 37 )$$

where v is a power that determines the strength of selection. One choice is v=2N−2, which assumes that the prevalence should be equal to the equilibrium frequency of the type in a haploid population in a Wright-Fisher process (maximum strength of selection), but other choices are possible. The parameter v can also be used to optimize the performance of the classifier. PWMs are then constructed according to Eqns. (34, 35) and so forth.

In FIGS. 2A-2C, the method for determining a function of a test sequence is set forth in further detail. A test sequence is sequence for which the function is unknown. The method is specifically illustrated as steps in FIG. 2C and pictorially in FIG. 2B. The blocks illustrated in the controller 14 perform the various functions as described below. In step 310, a plurality of sequences is provided to the controller 14 from the knowledge base 12B. The plurality of sequences may be referred to as an alignment of sequences. The functions associated with the sequences may be stored within the knowledge base 12B as described in step 312. While three PWM generators 18A-18n are illustrated, one PWM generator may be used for generating the PWMs of different orders. As mentioned above an alignment of sequences is usual but is not required. Alignment by motif is also possible.

In step 314, a first order PWM 410A (of FIG. 2) is generated at the PWM generator 18A using weights according to functional activity as in Eq. (37) at 410A. The first order PWM uses the position and the functions associated with the sequence. The first-order PWM and the formation thereof is described above.

In step 316, pairs of positions are used to create a second-order PWM. In step 318, a second order PWM 410B is generated at the PWM generator 18B using the pairs of positions.

In step 320, a third order PWM 410C is generated from triples of positions. In step 322, the third order PWM 210C is generated at the PWM generator 18C using the triples of positions. In step 324, nth-order multiplet positions are determined. In step 326, an nth-order PWM 210n is generated at the PWM generator 18n from nth-order multiplets of positions. In all of the steps 312 through 326, when the sequence is provided together with their function or functions, they are stored within the knowledge base 12B.

In step 328, for each sequence that has a measured function level, a sequence score is calculated in the sequence score generator 20. This is performed for all of the sequences and orders. The sequence score is calculated according to each order. The sequence score or IDSeq score may be plotted relative to the function level in step 330. FIG. 2 shows plots of the sequence score relative to the functions for each of the orders. That is, the first-order sequence score is plotted against the function in 420A, the second-order sequence score is plotted against the function in 420B, the third-order sequence score is plotted against the function in 420C and the sequence score is plotted against the function in 420n.

In step 332, a correlation coefficient is generated at the correlation generator 22. A correlation coefficient is a numerical measure of correlation, meaning a statistical relationship between two variables. Typically, the higher the value the higher the correlation. Typically, linear or nonlinear relationships between variables are indicated by a correlation coefficient. Typically, a value of 0.7 or greater indicates a strong correlation. A correlation coefficient is generated for each order. In step 334, the order with the highest correlation coefficient is selected. As indicated by the selection block 422C being a check mark (indicating the selected one) versus the selection blocks 422A, 422B and 422n being Xs.

In step 336, a regression is generated for the selected order. In this example, the third order correlation was the best and therefore regression is performed on the function versus third-order sequence score. In the present example, a linear regression is performed. However, other types of regression such as non-linear regression, may be used. By definition, non-linear regression includes linear regression. In step 338, a test sequence is obtained. The test sequence score is generated in step 340. In step 342, and as illustrated, the regression determined in step 336 is used to determine the function of the test sequence. That is, the sequence score of the test sequence is used as an input relative to the regression and therefore the function can be determined. For a linear regression, the sequence score R₃(s) is on the x axis when the sequence score is projected to the line 232, based upon the numerical value of the function, the predicted function is determined.

In FIGS. 1A and 2A, an adjustment block 50 may also be provided. The adjustment block 50 may have various components that are used to make adjustments to provide increased accuracy for the function prediction. The functions of each of the blocks of the adjustment block 50 is described in further detail below. The adjustment block 50 may include a reweighting block 52, an ambiguous state assignment resolver 54 and a strength of selection block 56, all of which are described in greater detail below.

If sequences in the knowledge base share common descent, the sequences in the knowledge base 12A, B are not in mutation-selection balance, which may confuse the IDSeq process. In such a case, it may be necessary to assign lower weight to the sequences with common descent in the reweighting block 52. There are many methods that can remove common descent from sequences in a knowledge base. In one method, the weight of a sequence is inversely proportional to the number of sequences that are within a fraction of the normalized Hamming distance to that sequence, depending on a similarity parameter θ. The choice of this similarity parameter can be made so as to optimize predictive performance.

Ambiguous state-assignments in the knowledge base 12 using weights may also be performed using the block 54. In many cases, sequences in databases contain symbols indicating that a state assignment is ambiguous, meaning that two or more symbols are equally likely at a particular position (according to the sequencing technology used). In the ambiguous state assignment resolver 54, sequence weights can be used to resolve ambiguous state assignments as follows. Suppose that at position i of sequence j in the knowledge base 12, both symbols A and B are indicated as equally likely. In this case, the count of symbol A at position i, n; (A) is increased by half the weight of sequence j: n(A)→n(A)+wⱼ/2 while the counter for symbol B also is increased by half the weight of sequence j: n(B)→n(B)+wⱼ/2. The effective size N_{eff} is unaffected. If there are 3 or more ambiguous states at the site, each counter is increased by a third of the weight, etc. When updating the counter of a pair of symbols (to calculate a 2nd-order PWM), then if there is symbol C at the second site), then the method adds wⱼ/2 to the AC counter, and wⱼ/2 to the BC counter.

The adjustment block 50 has a strength of selection block 56 to make an adjustment, which is described in more detail. The strength of selection can be modulated by assigning to each sequence j a weight given by the function fⱼ elevated to the power v

$${w}_{j} = \frac{{f}_{j}^{v}}{{\sum }_{k = 1}^{N} {f}_{k}^{{v}^{.}}} . \qquad ( 38 )$$

Note that this is the correct weight when highly functional sequences have high f. If instead a sequence has a low value f, then a new variable is created that has high value if f is low, for example e^{-f}. It is believed that in most cases a “fitness” measure may be determined given a measured quantity, and then a weight can be calculated from that.

A choice of v=0 implies not reweighting sequences according to fitness (no strength of selection). A choice of v=2N−2 implies choosing the maximum strength of selection of a haploid Wright-Fisher process. Intermediate choices of v may provide better algorithmic performance.

Example embodiments are provided so that this disclosure will be thorough and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A method comprising:
providing a plurality of sequences forming a knowledge base, each of the plurality of sequences having respective functions associated therewith;
forming a plurality of position weight matrices having different orders based on the sequences;
generating a sequence score for each of the plurality of sequences to form a plurality of sequence scores;
correlating the respective functions with the sequence scores to form correlation coefficients;
selecting a selected order from the different orders based on the correlation coefficients;
generating a test sequence score from a test sequence for the selected order; and
based on the test sequence score and the sequence scores, determining a function of the test sequence.

2. The method of claim 1 wherein determining the function of the test sequence comprises determining the function of the test sequence using regression.

3. The method of claim 1 wherein forming the plurality of position weight matrices having different orders comprises determining a first-order position weight matrix and a second-order position weight matrix.

4. The method of claim 3 wherein forming the plurality of position weight matrices having different orders further comprises determining a third-order position weight matrix.

5. The method of claim 4 wherein forming the plurality of position weight matrices having different orders further comprises determining a greater than third-order position weight matrix.

6. The method of claim 1 wherein providing the sequences comprise one of amino acid sequences, neural spike trains, and sequences written in any alphabet.

7. The method of claim 1 wherein providing the knowledge base sequences comprises providing nucleic acid sequences.

8. The method of claim 7 wherein after forming the plurality of position weight matrices, reweighting at least one of the plurality of position weight matrices to remove common ancestry.

9. The method of claim 7 wherein after forming the plurality of position weight matrices reweighting at least one of the plurality of position weight matrices to resolve ambiguous state assignments.

10. The method of claim 7 wherein after forming the plurality of position weight matrices reweighting at least one of the plurality of position weight matrices to create mutation-selection balance.

11. A system comprising:
a knowledge base having a plurality of sequences, each of the plurality of sequences having respective functions associated therewith; and
a controller programmed to
form a plurality of position weight matrices having different orders based on the sequences,
generate a sequence score for each of the plurality of sequences to form a plurality of sequence scores,
correlate the respective functions with the sequence scores to form correlation coefficients,
selecting a selected order from the different orders based on correlation coefficients,
generate a test sequence score from a test sequence for the selected order, and
based on the test sequence score and the sequence scores, determine a function of the test sequence.

12. The system of claim 11 wherein the controller is programmed to determine the function of the test sequence using regression.

13. The system of claim 11 wherein the plurality of position weight matrices comprises a first-order position weight matrix and a second-order weight position weight matrix.

14. The system of claim 11 wherein the plurality of position weight matrices comprises a first-order position weight matrix, a second-order weight position weight matrix and a third-order position weight matrix.

15. The system of claim 11 wherein the plurality of position weight matrices comprises a first-order position weight matrix, a second-order weight position weight matrix, a third-order position weight matrix and a greater than third-order weight matrix.

16. The system of claim 11 wherein the sequences comprise one of amino acid sequences, neural spike trains, or sequences written in any alphabet

17. The system of claim 11 wherein the sequences comprise nucleic acid sequences.

18. The system of claim 17 wherein the controller is programmed to reweight at least one of the plurality of position weight matrices to remove common ancestry.

19. The system of claim 17 wherein the controller is programmed to reweight at least one of the plurality of position weight matrices to resolve ambiguous state assignments.

20. The system of claim 17 wherein the controller is programmed to reweight at least one of the plurality of position weight matrices to adjust strength of selection.

21-25. (canceled)

26. The method of claim 1 wherein providing the sequences comprise one of amino acid sequences, neural spike trains, or sequences written in any alphabet.

27-41. (canceled)

42. A method comprising:
providing, in a knowledge base, a plurality of sequences having respective sequence scores and functions associated therewith;
generating a test sequence score; and
determining a function of the test sequence based on the test sequence score and the knowledge base sequence scores.

43. The method of claim 42 wherein determining the function of the test sequence comprises determining the function of the test sequence using regression.

44. The method of claim 42 wherein forming the plurality of position weight matrices having different orders comprises determining a first-order position weight matrix and a second-order weight position weight matrix.

45. The method of claim 44 wherein forming the plurality of position weight matrices having different orders further comprises determining a third-order position weight matrix.

46. The method of claim 45 wherein forming the plurality of position weight matrices having different orders further comprises determining a greater-than-third-order position weight matrix.

47. The method of claim 42 wherein providing the sequences comprise one of amino acid sequences, neural spike trains, or sequences written in any alphabet.

48. The method of claim 42 wherein providing the knowledge base sequences comprises providing nucleic acid sequences.

49. The method of claim 48 wherein after forming the plurality of position weight matrices, reweighting at least one of the plurality of position weight matrices to remove common ancestry.

50. The method of claim 48 wherein after forming the plurality of position weight matrices reweighting at least one of the plurality of position weight matrices to resolve ambiguous state assignments.

51. The method of claim 48 wherein after forming the plurality of position weight matrices reweighting at least one of the plurality of position weight matrices to adjust strength of selection.

52. A system comprising:
a knowledge base having a plurality of sequences, each of the plurality of sequences having respective functions associated therewith; and
a controller programmed to
generate a test sequence score; and
determining a function of the test sequence based on the test sequence score and the knowledge base sequence scores.

53. The system of claim 52 wherein the controller is programmed to determine the function of the test sequence using regression.

54. The system of claim 52 wherein the plurality of position weight matrices comprises a first-order position weight matrix and a second-order weight position weight matrix.

55. The system of claim 52 wherein the plurality of position weight matrices comprise a first-order position weight matrix, a second-order weight position weight matrix and a third order position weight matrix.

56. The system of claim 52 wherein the plurality of position weight matrices comprise a first-order position weight matrix, a second-order weight position weight matrix, a third-order position weight matrix and a greater-than-third-order position weight matrix.

57. The system of claim 52 wherein the sequences comprise one of amino acid sequences, neural spike trains, or sequences written in any alphabet.

58. The system of claim 52 wherein the sequences in the knowledge base comprise nucleic acid sequences.

59. The system of claim 58 wherein the controller is programmed to reweight at least one of the plurality of position weight matrices to remove common ancestry.

60. The system of claim 58 wherein the controller is programmed to reweight at least one of the plurality of position weight matrices to resolve ambiguous state assignments.

61. The system of claim 58 wherein the controller is programmed to reweight at least one of the plurality of position weight matrices to adjust strength of selection.
